(19) United States
(12) Patent Application Publication
(10) Pub. No.: US 2009/0035249 A1
(43) Pub. Date: Feb. 5, 2009

(54) Method of inhibiting proliferation of Escherichia coli

(76) Inventors: Sujata K. Bhatia, Newark, DE (US); Henry Keith Chenault, Hockessin, DE (US)

Correspondence Address: E I DU PONT DE NEMOURS AND COMPANY;LEGAL PATENT RECORDS CENTER, BARLEY MILL PLAZA 25/1122B, 4417 LANCASTER PIKE, WILMINGTON, DE, 19805, US

(21) Appl. No.: 12/220,743

(22) Filed: Jul. 28, 2008

Related U.S. Application Data
(60) Provisional application No. 60/963,026, filed on Aug. 2, 2007.

(51) Int. Cl. A61K 31/785; A61P 31/04

(52) U.S. Cl. 424/78.07

(57) ABSTRACT

A method of inhibiting proliferation of Escherichia coli at an anatomical site on tissue of a human or animal body is described. The method comprises applying to the site a bacteriostatically effective amount of an aminated polyether.

### CROSS-REFERENCE TO RELATED APPLICATION

This application claims priority under 35 U.S.C. §119 from U.S. Provisional Application Ser. No. 60/963,026, filed Aug. 2, 2007.

### FIELD OF THE INVENTION

The invention relates to the field of antimicrobial agents. More specifically, the invention relates to a method of inhibiting proliferation of Escherichia coli at an anatomical site on tissue of a human or animal body.

### BACKGROUND OF THE INVENTION

Various antimicrobial agents have been used to inhibit growth of harmful microorganisms in the treatment of wounds. Examples of antimicrobial agents include iodine formulations (e.g., Betadine® microbicides), mercury compounds (e.g. Merthiolate® antiseptic and Mercurochrome®), various silver salts (e.g., silver sulfadiazine), and numerous antibiotics.

Certain quaternary amine salts are also known to possess antimicrobial properties (e.g., benzalkonium chloride). Quaternary amines have also been incorporated into polymeric substrates to provide antimicrobial activity. For example, Perrault et al. (U.S. Pat. No. 6,800,278) describe an inherently antimicrobial quaternary amine wound dressing which is a hydrogel comprising a cationic quaternary amine acrylate polymer. Some primary amines are also known to possess antimicrobial activity. For example, Ottersbach et al. (WO 00/69925) describe a method of producing inherently microbiocidal polymer surfaces by polymerizing aliphatically unsaturated monomers that are at least singly functionalized by a primary amine group.

Tissue adhesives useful for various medical applications, such as wound closure, supplementing or replacing sutures or staples in internal surgical procedures, adhesion of synthetic onlays or inlays to the cornea, drug delivery devices, and as anti-adhesion barriers to prevent post-surgical adhesions, have been described. Antimicrobial agents are frequently added to these tissue adhesive compositions to inhibit bacterial growth at the site of application (see for example, Kodokian et al., copending and commonly owned U.S. Patent Application Publication No. 2006/0078536; and Rhee et al. U.S. Pat. No. 6,051,648).

There is a need for new methods of inhibiting growth of harmful microorganisms, such as Escherichia coli, for treatment of wounds caused by trauma or surgery. The method should comprise the use of an antimicrobial agent that is biocompatible and that can be applied in combination with various tissue sealants for use in wound closure and supplementing or replacing sutures or staples in internal surgical procedures (e.g., intestinal anastomosis), and in combination with antiadhesive polymers for adhesion prevention following surgery. The stated need is addressed herein by the discovery that aminated polyethers inhibit the growth of Escherichia coli.

### SUMMARY OF THE INVENTION

The invention provides a method of inhibiting proliferation of Escherichia coli suspected of being present at an anatomical site on tissue of a human or animal body comprising:

applying to said anatomical site a bacteriostatically effective amount of at least one aminated polyether.

### DETAILED DESCRIPTION OF THE INVENTION

Disclosed herein is a method of inhibiting proliferation of Escherichia coli at an anatomical site on tissue of a human or animal body that comprises applying an aminated polyether to the site. It was surprisingly discovered that aminated polyethers, which are non-toxic to human cells (Kodokian et al., supra), inhibit proliferation of Escherichia coli. The method may be used to prevent infection from wounds resulting from trauma or surgery and may be used in combination with tissue sealants for use in wound closure and supplementing or replacing sutures or staples in internal surgical procedures (e.g., intestinal anastomosis), and in combination with antiadhesive polymers for adhesion prevention following surgery.

The following definitions are used herein and should be referred to for interpretation of the claims and the specification.

The term “aminated polyether” refers to a polyether that contains at least two primary amine groups, and which is water soluble or able to be dispersed in water to form a colloidal suspension.

The term “polyether” refers to a polymer having the repeat unit [—O—R]—, wherein R is a hydrocarbylene group having 2 to 5 carbon atoms.

The term “multi-arm polyether amine” refers to a branched polyether, wherein at least two of the branches (“arms”) are terminated by a primary amine group, which is water soluble or able to be dispersed in water to form a colloidal suspension.

The term “branched polyether” refers to a polyether having one or more branch points (“arms”), including star, dendritic, comb, highly branched, and hyperbranched polyethers.

The term “dendritic polyether” refers to a highly branched polyether having a branching structure that repeats regularly with each successive generation of monomer, radiating from a core molecule.

The term “comb polyether” refers to a multi-arm polyether in which linear side chains emanate from trifunctional branch points on a linear polymer backbone.

The term “star polyether” refers to a multi-arm polyether in which linear side chains emanate from a single atom or a core molecule having a point of symmetry.

The term “highly branched polyether” refers to a multi-arm polyether having many branch points, such that the distance between branch points is small relative to the total length of the arms.

The term “hyperbranched polyether” refers to a multi-arm polyether that is more branched than highly branched with order approaching that of an imperfect dendritic polyether.

The term “branched end polyether amine” refers to a linear or multi-arm polyether having two or three primary amine groups at each of the ends of the polymer chain or at the end of the polymer arms.

The term “inhibiting proliferation of Escherichia coli” means—in the context of the present invention—that when an aminated polyether is applied to an anatomical site on tissue of a human or animal body, it will result in a decrease in the growth (i.e. the rate at which a population of cells increases) of the bacterium Escherichia coli at the site. “Inhibiting proliferation of Escherichia coli” does not require that the growth of the bacterium Escherichia coli at the site be totally prevented. Instead, “inhibiting proliferation of Escherichia coli” refers to a clinically significant reduction in the growth of Escherichia coli at the site, such that the likelihood of complications due to Escherichia coli infection are reduced significantly or eliminated.

The term “bacteriostatically effective amount” refers to the amount of aminated polyether that produces a 0.5 log decrease in bacterial growth of an Escherichia coli culture. The bacteriostatically effective amount of aminated polyethers is determined as described in Examples 1-5 herein.

The term “anatomical site” refers to any external or internal part of the body of humans or animals.

The term “tissue” refers to any tissue, both living and dead, in humans or animals.

The term “hydrogel” refers to a water-swellable polymeric matrix, consisting of a three-dimensional network of macromolecules held together by covalent or non-covalent crosslinks, that can absorb a substantial amount of water to form an elastic gel.

The term “crosslink” refers to a bond or chain attached between and linking two different polymer chains.

By medical application is meant medical applications as related to humans and for veterinary purposes.

### Aminated Polyethers:

The term “aminated polyether”, as used herein, refers to a polyether having the repeat unit [—O—R]— and at least two primary amine groups, wherein R is a hydrocarbylene group having 2 to 5 carbon atoms. The term “hydrocarbylene group” refers to a divalent group formed by removing two hydrogen atoms, one from each of two different carbon atoms, from a hydrocarbon. The aminated polyethers are water soluble or are able to be dispersed in water to form a colloidal suspension. Typically, aminated polyethers, like other polymers, are a heterogeneous mixture having a distribution of different molecular weights, and are characterized by an average molecular weight, for example, the weight-average molecular weight (M_{w}), or the number average molecular weight (Mₙ), as is known in the art. Suitable aminated polyethers have a number-average molecular weight of about 450 to about 200,000 Daltons, preferably from about 2,000 to about 40,000 Daltons.

The aminated polyether may be linear or branched. Suitable linear aminated polyethers include, but are not limited to, linear polyethylene oxides (also referred to herein as polyethylene glycols or PEGs), linear polypropylene oxides, and linear polyethylene oxide-polypropylene oxide copolymers, having primary amine groups on each end of the polymer chain. Suitable branched aminated polyethers, also referred to herein as multi-arm polyether amines, include, but are not limited to, dendritic, comb, star, highly branched, and hyperbranched polyethers wherein at least two of the arms are terminated by a primary amine group. Examples of multi-arm polyether amines include, but are not limited to, amino-terminated star, dendritic, or comb polyethylene oxides; amino-terminated star, dendritic or comb polypropylene oxides; amino-terminated star, dendritic or comb polyethylene oxide-polypropylene oxide copolymers; and polyoxyalkylene triamines, sold under the trade name Jeffamine® triamines, by Huntsman LLC. (Houston, Tex.). Examples of star polyethylene oxide amines, include, but are not limited to, various multi-arm polyethylene glycol amines, available from Nektar Transforming Therapeutics (Huntsville, Ala.), and star polyethylene glycols having 3, 4, 6 or 8 arms terminated with primary amines (referred to herein as 3, 4, 6 or 8-arm PEG amines, respectively). Examples of suitable Jeffamine® triamines include, but are not limited to, Jeffamine® T-403 (CAS No. 39423-51-3), Jeffamine® T-3000 (CAS No. 64852-22-8), and Jeffamine® T-5000 (CAS No. 64852-22-8).

Suitable aminated polyethers are either available commercially, as noted above, or may be prepared using methods known in the art. For example, linear polyethylene glycol diamines can be prepared by putting amine ends on linear polyethylene glycols, which are available from companies such as Sigma-Aldrich (Milwaukee, Wis.) and Fluka Chemical Corp. (Milwaukee, Wis.). Similarly, multi-arm polyethylene glycol amines can be prepared by putting amine ends on multi-arm polyethylene glycols (e.g., 3, 4, 6, and 8-arm star polyethylene glycols, available from companies such as Nektar Transforming Therapeutics; SunBio, Inc., Anyang City, South Korea; NOF Corp., Tokyo, Japan; or JenKem Technology USA, Allen, Tex.). The polyethylene glycols can be converted to polyethylene glycol amines using the method described by Buckmann et al. (Makromol. Chem. 182:1379-1384, 1981). In that method, the polyethylene glycol is reacted with thionyl bromide to convert the hydroxyl groups to bromines, which are then converted to amines by reaction with ammonia at 100° C. The method is broadly applicable to the preparation of other aminated polyethers. Additionally, aminated polyethers can be prepared from polyethers using the method described by Chenault (copending and commonly owned U.S. Patent Application Publication No. 2007/0249870). In that method, the polyether is reacted with thionyl chloride to convert the hydroxyl groups to chlorine groups, which are then converted to amines by reaction with aqueous or anhydrous ammonia, as described in detail in the Examples herein below. Other methods that may be used for preparing aminated polyethers are described by Merrill et al. in U.S. Pat. No. 5,830,986, and by Chang et al. in WO 97/30103.

The aminated polyether can also be a branched end polyether amine, as described by Arthur (copending and commonly owned patent application Ser. No. 07/24393). The branched end polyether amines can be linear or branched polymers having two or three amine groups at each of the ends of the polymer chain or at the end of the polymer arms. The starting materials used to prepare the branched end polyether amines can be linear polymers such as polyethylene oxide, poly(trimethyleneoxide), block or random copolymers of polyethylene oxide and polypropylene oxide or triblock copolymers of polyethylene oxide and polypropylene oxide, having terminal hydroxyl groups, or branched polymers such as multi-arm polyethers including, but not limited to, comb and star polyethers. The branched end polyether amines can be prepared by attaching multiple amine groups to the ends of the polymer by reaction with the hydroxyl groups using methods known in the art. For example, a branched end polyether amine having two amine functional groups on each end of the polymer chain or at the end of the polymer arms can prepared by reacting the starting material, as listed above, with thionyl chloride in a suitable solvent such as toluene to give the chloride derivative, which is subsequently reacted with tris(2-aminoethyl)amine to give the branched end reactant having two amino groups at each end of the polymer chain or arm, as described in detail in the Examples below.

In one embodiment the aminated polyether is selected from the group consisting of a linear polyethylene glycol diamine having a number-average molecular weight of about 2,000 Daltons (referred to herein as P2-2-1), an eight arm polyethylene glycol octaamine having a number-average molecular weight of about 10,000 Daltons (referred to herein as P8-10-1), an eight arm polyethylene glycol octaamine having a number-average molecular weight of about 2,000 Daltons (referred to herein as P8-2-1), a four arm polyethylene glycol tetraamine having a number-average molecular weight of about 2,000 Daltons (referred to herein as P4-2-1), an 8-arm polyethylene glycol hexadecaamine having a number-average molecular weight of about 40,000 Daltons (referred to herein as P8-40-2), an 8-arm polyethylene glycol hexadecaamine having a number-average molecular weight of about 10,000 Daltons (referred to herein as P8-10-2), and any combination thereof. In one embodiment, the aminated polyether is an eight arm polyethylene glycol octaamine having a number-average molecular weight of about 2,000 Daltons (P8-2-1).

It should be recognized that the multi-arm polyether amines are generally a somewhat heterogeneous mixture having a distribution of arm lengths and in some cases, a distribution of species with different numbers of arms. When a multi-arm polyether amine has a distribution of species having different numbers of arms, it can be referred to based on the average number of arms in the distribution. For example, in one embodiment the multi-arm polyether amine is an 8-arm star PEG amine, which comprises a mixture of multi-arm star PEG amines, some having less than and some having more than 8 arms; however, the multi-arm star PEG amines in the mixture have an average of 8 arms. Therefore, the terms “8-arm”, “6-arm”, “4-arm” and “3-arm” as used herein to refer to multi-arm polyether amines, should be construed as referring to a heterogeneous mixture having a distribution of arm lengths and in some cases, a distribution of species with different numbers of arms, in which case the number of arms recited refers to the average number of arms in the mixture.

In one embodiment, the aminated polyether is contained in an aqueous solution or dispersion. To prepare the aqueous solution or dispersion comprising the aminated polyether, at least one aminated polyether is added to water to give a concentration which comprises a bacteriostatically effective amount of the aminated polyether. A combination of different aminated polyethers can also be used. As used herein, a bacteriostatically effective amount refers to the amount of aminated polyether that produces a 0.5 log decrease in bacterial growth of an Escherichia coli culture. As described in detail in Examples 1-5 below, the bacteriostatically effective amount is determined by seeding cultures of Escherichia coli in a suitable growth medium, such as Luria Broth, with varying amounts of the aminated polyether. The cultures are then incubated for a sufficient period of time for the cultures to grow, typically overnight. Then, bacterial growth is determined using methods known in the art, for example by optical density measurement or by plating the cultures and counting the colony forming units (CFUs) on the plates.

For use on living tissue, it is preferred that the aqueous solution or dispersion comprising the aminated polyether be sterilized. Any suitable sterilization method known in the art may be used, including, but not limited to, electron beam irradiation, gamma irradiation, ethylene oxide sterilization, or ultra-filtration through a 0.2 μm pore membrane.

The aqueous solution or dispersion comprising the aminated polyether may further comprise various additives depending on the intended application. For example, the aqueous solution or dispersion comprising the aminated polyether may include at least one additive selected from the group consisting of thickeners, colorants, surfactants, and anti-inflammatory agents. The amount of the additive used depends on the particular application and may be readily determined by one skilled in the art using routine experimentation.

The aqueous solution or dispersion comprising the aminated polyether may optionally include at least one thickener. The thickener may be selected from among known viscosity modifiers, including, but not limited to, polysaccharides and derivatives thereof, such as starch or hydroxyethyl cellulose.

The aqueous solution or dispersion comprising the aminated polyether may also optionally include at least one colorant to enhance the visibility of the solution. Suitable colorants include dyes, pigments, and natural coloring agents. Examples of suitable colorants include, but are not limited to, FD&C and D&C colorants, such as FD&C Violet No. 2, D&C Green No. 6, D&C Green No. 5, D&C Violet No. 2; and natural colorants such as beetroot red, canthaxanthin, chlorophyll, eosin, saffron, and carmine.

The aqueous solution or dispersion comprising the aminated polyether may also optionally include at least one surfactant. Surfactant, as used herein, refers to a compound that lowers the surface tension of water. The surfactant may be an ionic surfactant, such as sodium lauryl sulfate, or a neutral surfactant, such as polyoxyethylene ethers, polyoxyethylene esters, and polyoxyethylene sorbitan.

Additionally, the aqueous solution or dispersion comprising the aminated polyether may optionally include anti-inflammatory agents, such as indomethacin, salicylic acid acetate, ibuprophen, sulindac, piroxicam, and naproxen; and thrombogenic agents, such as thrombin, fibrinogen, homocysteine, and estramustine.

Method of Inhibiting Proliferation of Escherichia coli

The method disclosed herein for inhibiting proliferation of Escherichia coli suspected of being present at an anatomical site on tissue of a human or animal body comprises: applying to the site a bacteriostatically effective amount of at least one aminated polyether. In one embodiment, the bacteriostatically effective amount of at least one aminated polyether is applied in the form of an aqueous solution or dispersion, as described above. The solution may be applied to the site using any suitable method including, but not limited to, brushing, pouring, dripping, spraying, wiping, or extrusion using a pipette or a syringe. Additionally, the aminated polyether may be applied to the site as a controlled release formulation using a controlled release drug delivery system, as is well known in the art (see for example, Encyclopedia of Controlled Drug Delivery, Volumes 1 and 2, E. Mathiowitz, ed., 1999, John Wiley & Sons, New York).

The method disclosed herein has many potential medical and veterinary applications. The method would be useful in applications where the inhibition of Escherichia coli proliferation is required to prevent medical complications. Examples include, but are not limited to, treatment of wounds caused by trauma or surgery, internal surgical procedures such as intestinal anastomosis and vascular anastomosis, and anti-adhesive applications.

One specific example is the use of the method in combination with a tissue sealant in supplementing or replacing sutures or staples in an intestinal anastomosis procedure. In that application, the aminated polyether may be applied to the intestines around the sutures or staples and then a tissue sealant may be applied to the site to seal the sutures or staples. In this way, the proliferation of Escherichia coli at the site may be inhibited, thereby decreasing the risk of abscess formation. Any suitable tissue sealant known in the art may be used. For example, hydrogel sealants, such as those described by Rhee et al. (U.S. Pat. No. 6,534,591) and Kodokian et al., supra, which are formed by reacting a component having nucleophilic groups with a component having electrophilic groups, which are capable of reacting with the nucleophilic groups of the first component, to form a crosslinked network via covalent bonding. If the electrophilic groups are capable of reacting with amine groups and therefore would react with the amine groups of the aminated polyether, the concentration of the components is chosen such that there is an excess of amine groups on the hydrogel that is formed.

Additionally, it is anticipated that the aminated polyether may be applied as a coating on a medical implant. For example, the aminated polyether could be coated onto or impregnated into sutures or coated onto staples to provide a bacteriostatic coating.

The method disclosed herein may also be used in combination with an antiadhesive polymer in preventing post surgical adhesions. In that application, the aminated polyether may be applied to the surgical site before the application of the antiadhesive polymer to inhibit proliferation of Escherichia coli at the site and reduce the risk of abscess formation. The antiadhesive polymer may be any suitable polymer having antiadhesive properties, including but not limited to hydrogel sealants, such as those described by Rhee et al. (U.S. Pat. No. 6,534,591, and U.S. Patent Application Publication No. 2004/0235708), Sawhney (U.S. Patent Application Publication No. 2003/0108511) and Kodokian et al. supra, or a polyoxyalkylene block copolymer, as described by Henry et al. (U.S. Pat. No. 4,911,926).

### EXAMPLES

The present invention is further defined in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various uses and conditions.

The meaning of abbreviations used is as follows: The meaning of abbreviations used is as follows: “min” means minute(s), “h” means hour(s), “sec” means second(s), “mL” means milliliter(s), “L” means liter(s), “μL” means microliter(s), “cm” means centimeter(s), “mm” means millimeter(s), “μm” means micrometer(s), “mol” means mole(s), “mmol” means millimole(s), “g” means gram(s), “kg” means kilogram(s), “mg” means milligram(s), “Pa” means pascal(s), “kPa” means kilopascal(s), the designation “10K” means that a polymer molecule possesses a weight-average molecular weight of 10 kiloDaltons, a designation of “60K” indicates a weight-average molecular weight of 60 kiloDaltons, etc., “NMR” means nuclear magnetic resonance spectroscopy, “M” means molar concentration, “Mₙ” means number-average molecular weight, “wt %” means percent by weight, “PEG” means polyethylene glycol. A reference to “Aldrich” or a reference to “Sigma” means the said chemical or ingredient was obtained from Sigma-Aldrich, St. Louis, Mo.

### General Methods:

### Reagents:

The 8-arm PEG (Mₙ=10,000, referred to herein as “8-arm PEG 10K”), having eight arms, each terminated by a hydroxyl group, and the 4-arm PEG (Mₙ=2,000, referred to herein as “4-arm PEG 2K”), having four arms, each terminated by a hydroxyl group, and the 8-arm PEG (Mₙ=2,000, referred to herein as “8-arm PEG 2K”), having eight arms, each terminated by a hydroxyl group, were purchased from NOF America Corp. (White Plains, N.Y.) or Shearwater Polymers Inc. (Huntsville, Ala.), as noted below. The linear PEG (Mₙ=2,000), referred to herein as “PEG 2K”, was purchased from Fluka Chemical Corp. (Milwaukee, Wis.).

### Preparation of Peg Amines:

Preparation of 8-Arm Polyethylene Glycol 10K Octaamine:

An 8-arm PEG 10K octaamine, referred to herein as “P8-10-1,” was synthesized using the two-step procedure described by Chenault in co-pending and commonly owned U.S. Patent Application Publication No. 2007/0249870. A typical synthesis is described here. In the first step, the 8-arm PEG 10K was converted to an 8-Arm PEG 10K chloride by reaction with thionyl chloride, i.e.,

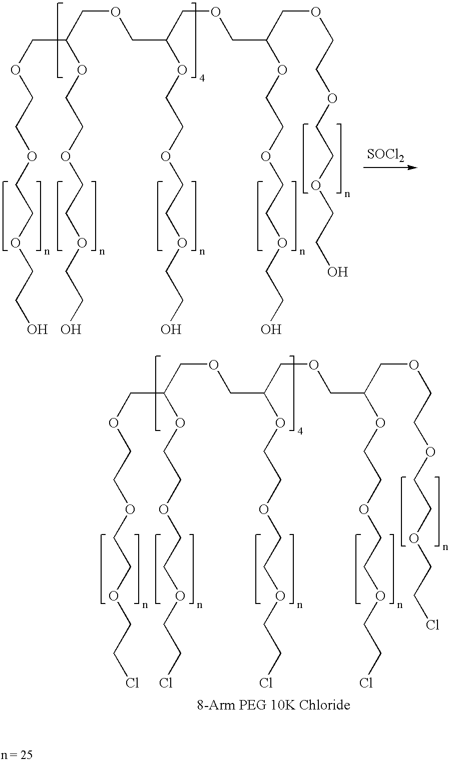

The 8-arm PEG 10K (NOF Sunbright HGEO-10000; 1000 g in a 3-L round-bottom flask) was dried by dissolving it in 1.5 L of toluene and distilling 500 mL of toluene-water azeotrope plus toluene under reduced pressure (2 kPa) with a pot temperature of 60° C., adding another 500 mL of toluene to the pot, and distilling 500 mL of toluene-water azeotrope plus toluene under reduced pressure (2 kPa) with a pot temperature of 60° C.

The solution of 8-arm PEG was allowed to cool to room temperature. Then, thionyl chloride (233 mL, 3.19 mol) was added to the flask, which was equipped with a reflux condenser, and the mixture was heated at 85° C. with stirring under a blanket of nitrogen for 4 h. Excess thionyl chloride and most of the toluene were removed by vacuum distillation at 2 kPa (bath temp 40-60° C.). Two successive 500-mL portions of toluene were added and evaporated under reduced pressure (2 kPa, bath temperature 80-85° C.) to complete the removal of thionyl chloride. The final crude product was dissolved in 1000 g of de-ionized water.

In the second step, the 8-Arm PEG 10K chloride was converted to the 8-Arm PEG 10K amine by reaction with aqueous ammonia, i.e.,

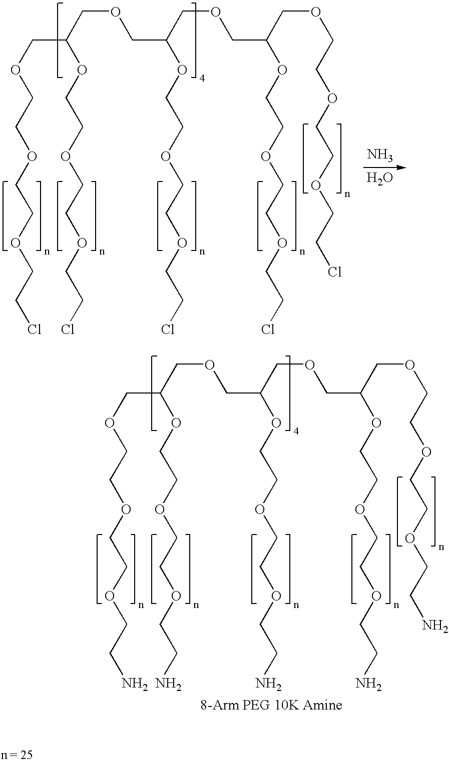

The aqueous solution of 8-arm PEG-Cl prepared above, was dissolved in 16 L of concentrated aqueous ammonia (28 wt %) and heated in a sealed stainless steel pressure vessel at 60° C. for 48 h. The solution was sparged for 24 h with dry nitrogen and then placed under reduced pressure for 3 h to drive off ammonia. The solution was then passed through a column of strongly basic anion exchange resin (5 kg; Purolite® A-860, The Purolite Co., Bala-Cynwyd, Pa.) in the hydroxide form. The eluant was collected, and two 7-L portions of de-ionized water were passed through the column and collected. The aqueous fractions were combined, concentrated under reduced pressure (2 to 0.3 kPa, bath temperature 60° C.) to give the 8-Arm PEG 10K octaamine. The final product was characterized by proton NMR and size exclusion chromatography (SEC), as described by Chenault, supra.

Preparation of 4-Arm Polyethylene Glycol 2K Tetraamine:

A 4-arm PEG 2K tetraamine, referred to herein as “P4-2-1,” was prepared using a similar procedure as described above for the 8-arm PEG 10K octaamine. A typical synthesis is described here. In the first step, the 4-arm PEG 2K was converted to a 4-arm PEG 2K chloride by reaction with thionyl chloride, i.e.,

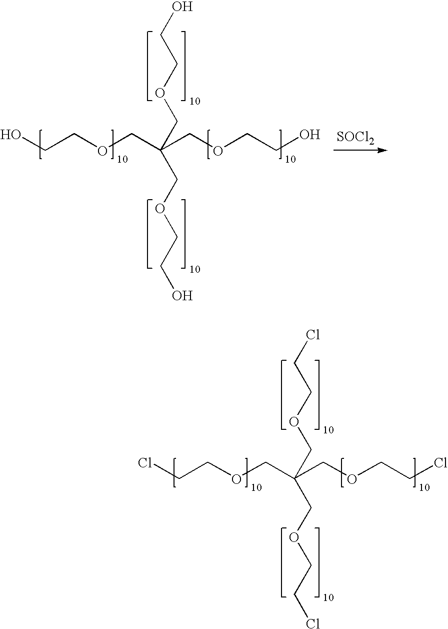

The 4-arm PEG 2K (NOF Sunbright PTE-2000; 1000 g in a 3-L round-bottom flask) was dried by dissolving it in 600 mL of toluene and distilling off the toluene-water azeotrope plus toluene under reduced pressure (2 kPa) with a pot temperature of 42° C., adding another 600 mL of toluene to the pot, and repeating the azeotropic distillation. The dried 4-arm PEG was dissolved in 1000 mL of toluene and 1.50 g (20 mmol) of N,N-dimethylformamide, and the solution was warmed to 60-65° C. Then, thionyl chloride (584 mL, 8.00 mol) was added to the flask, which was equipped with a reflux condenser, and the mixture was heated at 85° C. with stirring under a blanket of nitrogen for 2 h. Excess thionyl chloride and most of the toluene were removed by vacuum distillation at 2 kPa (bath temp 40° C.). Two successive 600-mL portions of toluene were added and evaporated under reduced pressure (2 to 0.3 kPa, bath temperature 40-85° C.) to complete the removal of thionyl chloride.

In the second step, the 4-arm PEG 2K chloride was converted to the 4-arm PEG 2K amine by reaction with aqueous ammonia, i.e.,

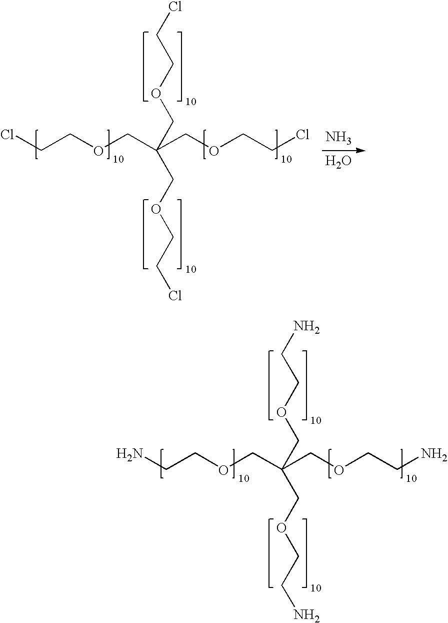

The 4-arm PEG-Cl (1000 g) was dissolved in 16 L of concentrated aqueous ammonia (28 wt %) and heated in a sealed stainless steel pressure vessel at 60° C. for 48 h. The solution was sparged for 24 h with dry nitrogen and then placed under reduced pressure for 2 h to drive off ammonia. The solution was then passed through a column of strongly basic anion exchange resin (5 kg, Purolite® A-860) in the hydroxide form. The eluant was collected, and two 7-L portions of de-ionized water were passed through the column and collected. The aqueous fractions were combined, concentrated under reduced pressure (2 to 0.3 kPa, bath temperature 60° C.) to give the 4-arm PEG 2K tetraamine. The final product was characterized by proton NMR and size exclusion chromatography (SEC), as described by Chenault, supra.

Preparation of 8-Arm PEG 40K Hexadecaamine:

An 8-arm PEG 40K hexadecaamine, referred to herein as “P8-40-2,” was prepared using a similar procedure as described above for the 8-arm PEG 10K octaamine, except that the 8-arm PEG chloride formed in the first step was reacted with tris(2-aminoethyl)amine to give the 8-arm PEG 40K hexadecaamine. A typical synthesis is described here.

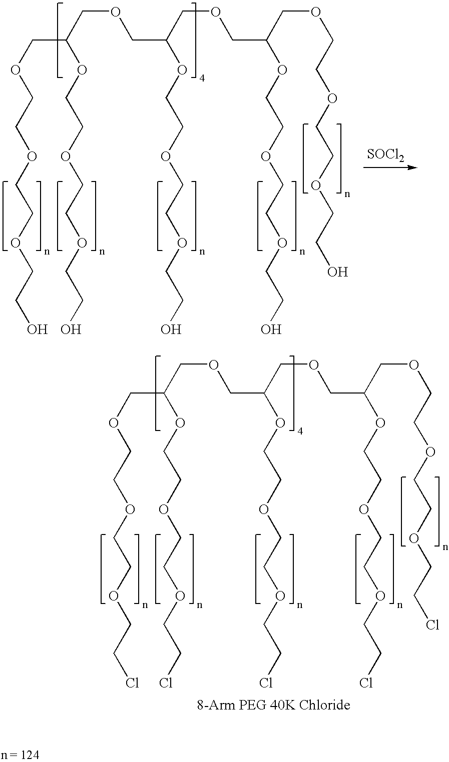

The 8-arm PEG 40K (NOF Sunbright HGEO-40000; 1000 g in a 4-L resin kettle) was dried by dissolving it in 1.5 L of toluene and distilling 500 mL of toluene-water azeotrope plus toluene under reduced pressure with a pot temperature of 70-80° C. After the distillation, the vacuum was broken with nitrogen, and 1.5 g (20 mmol) of N,N-dimethylformamide was added. Then, thionyl chloride (59 mL, 0.80 mol) was added to the reactor, which was equipped with a reflux condenser, and the mixture was heated at 85° C. with stirring under a blanket of nitrogen for 4 h. The reaction mixture was diluted with 1 L of toluene, held at 60-70° C., and then transferred into a 12-L, round-bottom flask equipped with an overhead stirrer and containing 4 L of heptane. The mixture was stirred under nitrogen and allowed to cool to room temperature. The resulting slurry was filtered, and the recovered solids were washed with 500 mL of heptane and then dried with aspiration under a flow of dry nitrogen to give the 8-arm PEG 40K chloride.

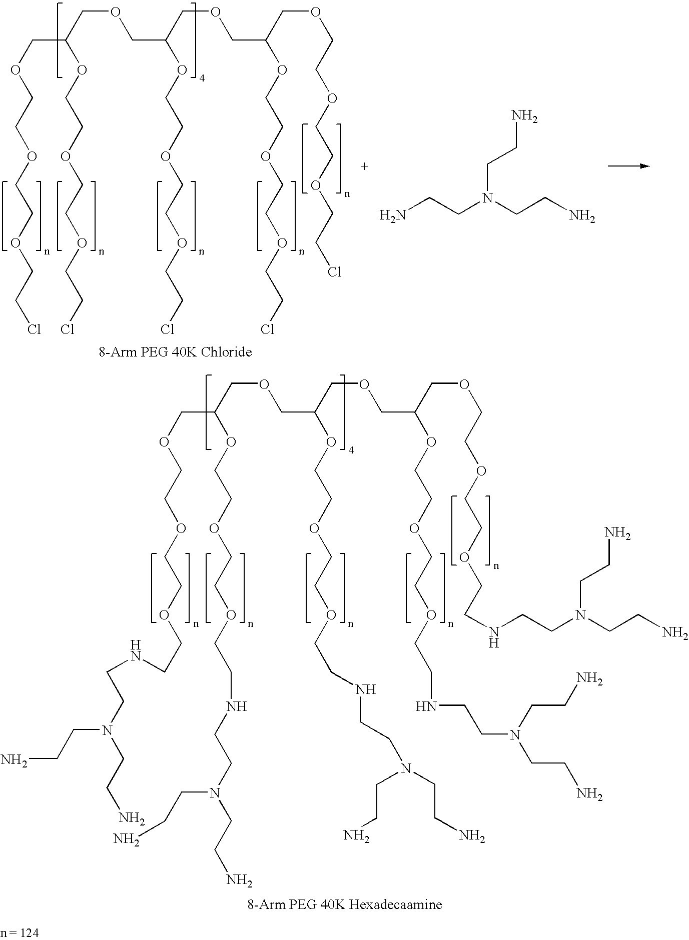

A 12-L, 4-neck round-bottom flask equipped with a mechanical stirrer and reflux condenser was charged with 993 g of 8-arm star PEG 40K chloride and 2000 mL of water. The mixture was heated to 90° C. with stirring to dissolve the PEG chloride. Then, 1200 mL (8.0 mol) of tris(2-aminoethyl)amine (TCI America, Portland, Oreg.; #T1243) was added. The reaction mixture was stirred at 100° C. for 8 h and then allowed to cool to room temperature overnight under nitrogen. The solution was transferred to a 20-L vessel under nitrogen and extracted with 10 L of dichloromethane. The organic layer was collected, dried over sodium sulfate with stirring under nitrogen, and then filtered through a pad of Celite® diatomaceous earth (World Minerals, Lompoc, Calif.). The solution was concentrated to about 2 L and poured slowly with stirring into a 20-L vessel containing 10 L of methyl t-butyl ether. The resulting slurry was cooled to 0° C. and then filtered. The recovered solids were washed with 1.0 L of cold methyl t-butyl ether and then dried under vacuum to give the P8-40-2. The final product was characterized by proton NMR and size exclusion chromatography (SEC), as described by Chenault, supra.

Preparation of 8-Arm PEG 10K Hexadecaamine:

An 8-arm PEG 10K hexadecaamine, referred to herein as “P8-10-2”, was prepared using a two step procedure in which 8-arm PEG 10K was reacted with methanesulfonyl chloride in dichloromethane in the presence of triethylamine to produce 8-arm PEG 10K mesylate, which was subsequently reacted with tris(2-aminoethyl)amine to give the 8-arm PEG 10K hexadecaamine. A typical synthesis is described here.

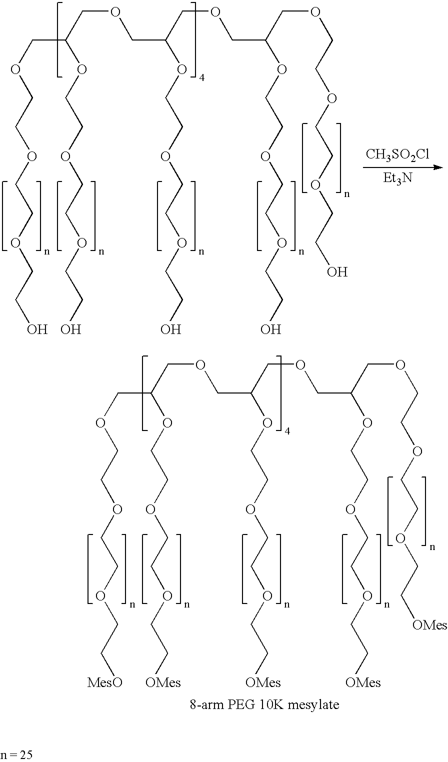

A solution of 10 g (8.0 mmol OH) of 8-arm PEG 10K (Mₙ=10,000; Shearwater Polymers Inc., Huntsville, Ala.) in 140 mL of dichloromethane was stirred under nitrogen as 4 mL (28 mmol) of triethylamine (Et₃N) was added, followed by 2.6 g (1.8 mL; 24 mmol) of methanesulfonyl chloride. The mixture was stirred at room temperature over the weekend.

Then, the solution was gently stirred with three 25-mL portions of 10% sodium carbonate for 15 min each. However, separation was difficult, so the whole mixture was placed in dry ice to freeze the water and allow the dichloromethane to be decanted away. The dichloromethane was dried with magnesium sulfate, filtered through Celite® diatomaceous earth (World Minerals, Lompoc, Calif.), concentrated to about 15 mL and added to 600 mL of diethyl ether with stirring. The ether was stirred in an ice bath for 20 min and the suspension was vacuum filtered under nitrogen, taken up in dichloromethane and reprecipitated into 600 mL of fresh ether. The suspension was chilled and vacuum filtered under nitrogen followed by three ether washings of 20 mL each under nitrogen with careful exclusion of air to yield 7.94 g of 8-arm PEG 10K mesylate.

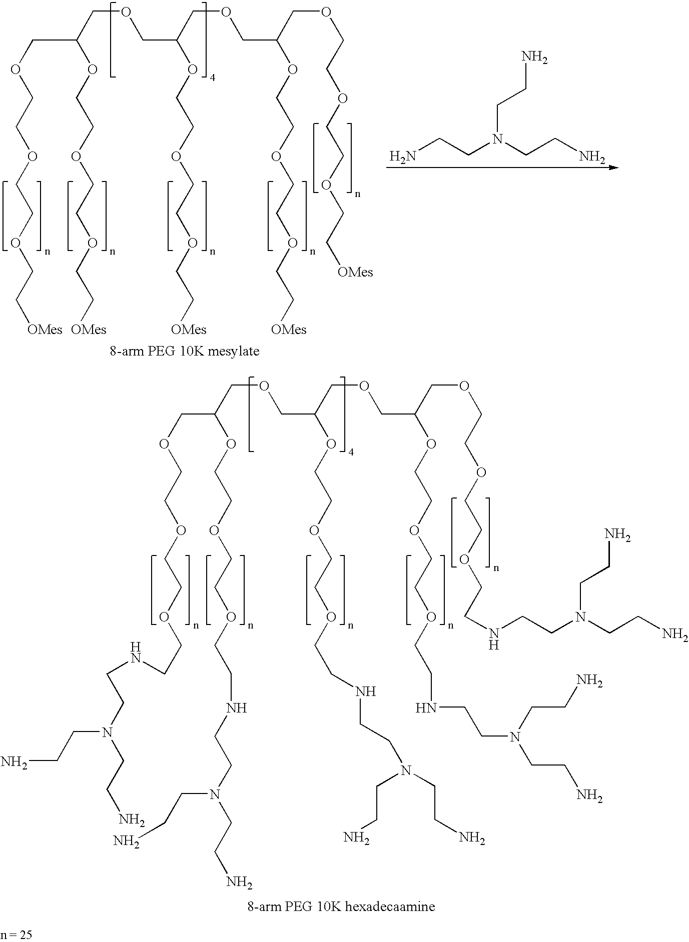

A solution of 2.0 g (2.2 mmol OMes) of 8-arm star PEG 10K mesylate in 10 mL of water was chilled in an ice bath and 10 mL (68 mmol) of tris(2-aminoethyl)amine (Aldrich # 225630) was added. The solution was stirred at room temperature for 96 h. Then, 1 mL of 10% sodium carbonate was added and the mixture was extracted with three 25-mL portions of dichloromethane with gentle stirring for 5 min. The dichloromethane layers were combined, dried with sodium sulfate, filtered and concentrated to about 15 mL. The concentrate was added with stirring to 250 mL of diethyl ether at room temperature. The ether was then stirred in an ice bath for 20 min and the resulting white precipitate was vacuum filtered under nitrogen. The product was dissolved on the funnel in 10 mL of dichloromethane in 3 portions and the solution was passed through into a 50-mL round bottom flask using vacuum. The solution was reprecipitated from 200 mL of more ether followed by chilling in ice and vacuum filtration under nitrogen to yield 1.55 g of the 8-arm PEG 10K hexadecaamine. The final product was characterized by proton NMR and size exclusion chromatography (SEC), as described by Chenault, supra.

Preparation of 8-Arm PEG 2K Octaamine

An 8-arm PEG 2K octaamine, referred to herein as “P8-2-1,” was prepared using a two step procedure in which 8-arm PEG 2K was reacted with methanesulfonyl chloride in dichloromethane in the presence of triethylamine to produce 8-arm PEG 2K mesylate, which was subsequently reacted with aqueous ammonia to give the 8-arm PEG 2K octaamine. A typical synthesis is described here.

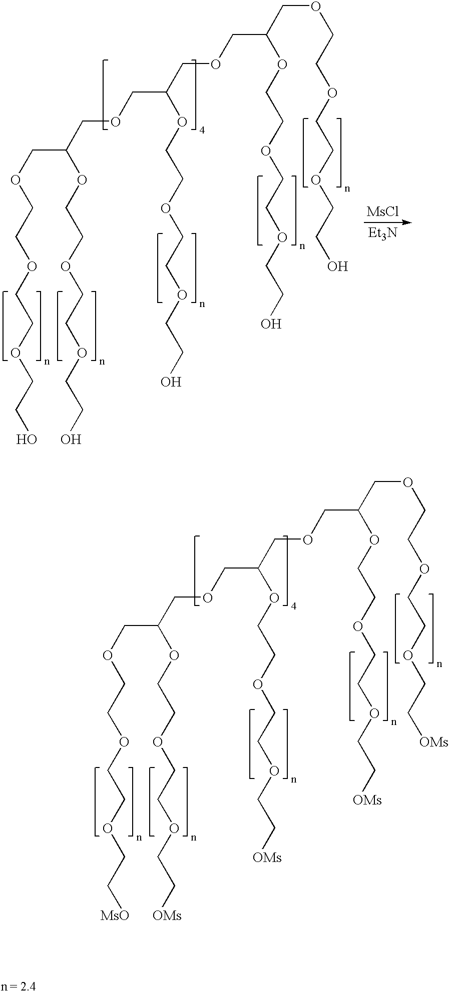

A solution of 10 g of 8-arm PEG 2K (NOF Sunbright HGEO-2000) in 235 mL of dichloromethane was stirred under nitrogen as 10.6 mL of triethylamine was added, followed by addition of 5.8 mL of methanesulfonyl chloride. The mixture was stirred at room temperature overnight. The reaction mixture was transferred to a separatory funnel and washed with 3×50 mL of 1.0 M potassium dihydrogen phosphate, 1×50 mL of 1.0 M potassium carbonate, and 1×50 mL of water. The dichloromethane layer was dried over magnesium sulfate, filtered, and concentrated under reduced pressure (2 kPa) with a pot temperature of 40° C. to give the 8-arm PEG 2K mesylate.

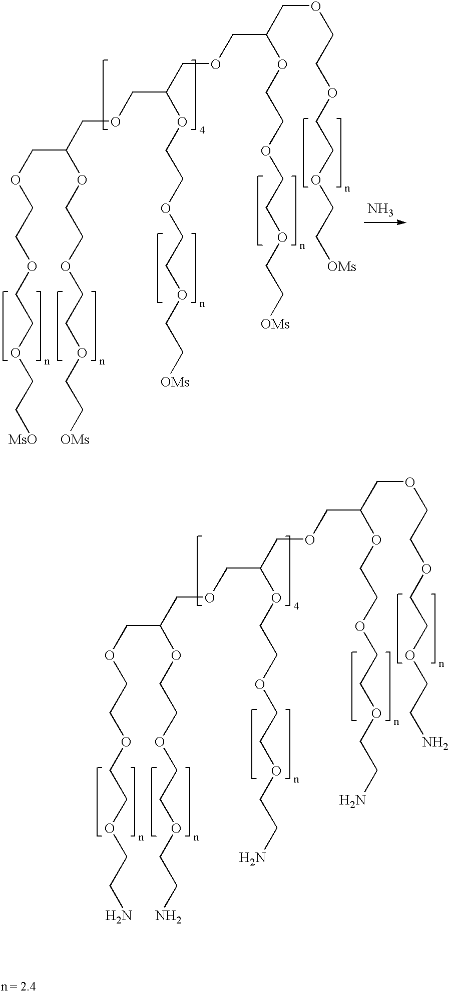

A solution of 12.33 g of 8-arm PEG 2K mesylate in 350 mL of concentrated aqueous ammonia (28 wt %) was stirred in a sealed vessel at room temperature for 48 h. The solution was sparged with dry nitrogen for 2 h. Then, 5 mL of 1.0 M potassium carbonate was added, and the solution was extracted with 3×50 mL of dichloromethane. The combined organic layers were dried over magnesium sulfate, filtered, and concentrated under reduced pressure (2 kPa) with a pot temperature of 40° C. to give the 8-arm PEG 2K octaamine. The final product was characterized by proton NMR and size exclusion chromatography (SEC), as described by Chenault, supra.

Preparation of Linear PEG 2K Diamine

An linear PEG 2K diamine, referred to herein as “P2-2-1,” was prepared using a two step procedure in which linear PEG 2K was reacted with methanesulfonyl chloride in dichloromethane in the presence of triethylamine to produce linear PEG 2K mesylate, which was subsequently reacted with aqueous ammonia to give the linear PEG 2K diamine. A typical synthesis is described here.

A solution of 20 g of linear PEG 2K (Mₙ=2,000, Fluka, #81221) in 280 mL of dichloromethane was stirred under nitrogen as 5.7 mL of triethylamine was added, followed by addition of 3.2 mL of methanesulfonyl chloride. The mixture was stirred at room temperature overnight. The reaction mixture was diluted with 100 mL of chloroform, transferred to a separatory funnel, and washed with 3×100 mL of 1.0 M potassium dihydrogen phosphate and 1×100 mL of 1.0 M potassium carbonate. The organic layer was dried over magnesium sulfate, filtered, and concentrated under reduced pressure (2 kPa) with a pot temperature of 40° C. to give linear PEG 2K mesylate.

A solution of 5 g of linear PEG 2K mesylate in 10 mL of acetonitrile and 40 mL of concentrated aqueous ammonia (28 wt %) was stirred in a sealed vessel at room temperature for 48 h. The solution was sparged with dry nitrogen for 2 h and then concentrated briefly under reduced pressure (2 kPa) with a pot temperature of 40° C. Then, 10 mL of 1.0 M potassium carbonate was added, and the solution was extracted with 3×25 mL of chloroform. The combined chloroform layers were dried over magnesium sulfate, filtered, and concentrated under reduced pressure (2 kPa) with a pot temperature of 40° C. to give the linear PEG 2K diamine. The final product was characterized by proton NMR and size exclusion chromatography (SEC), as described by Chenault, supra.

### Examples 1-5

### In Vitro Testing of PEG Amines for Inhibition of E. coli Growth

The purpose of these Examples was to demonstrate the antimicrobial activity of PEG amines by testing their inhibition of E. coli growth.

A suspension culture of E. coli (strain K12, ATCC No. 25257) from American Type Culture Collection (ATCC; Manassas, Va.) was prepared by seeding an individual colony into 4 mL of Luria Broth (obtained from ATCC). The culture was incubated in a shaker overnight at 37° C. to allow the culture to reach the saturation point of cell growth. The concentration of E. coli in the saturated overnight culture was approximately 1×10⁹ cells/mL. The saturated overnight culture (10 μL) was then seeded into 4 mL of Luria Broth, and the desired amount of a PEG amine was added, as indicated in Table 1. The culture was incubated in a shaker overnight at 37° C. Following the incubation, bacterial growth was quantified by measuring the optical density of the culture at 600 nm using a spectrophotometer. This method for determining in vitro antimicrobial activity has been shown to correlate with in vivo antimicrobial activity (Lee S H et al., Journal of Pharmacy and Pharmacology 2003 April; 55:559-66.)

The results are summarized in Table 1. The bacteriostatically effective amount of each PEG amine, i.e., the amount of the PEG amine that produced a 0.5 log decrease in bacterial growth, is also given in the Table. The bacteriostatically effective amount of each PEG amine was estimated from a plot of the log of bacterial growth (cells/mL) versus the volume of PEG amine added to the culture medium. The results indicate that all of the PEG amines inhibited E. coli growth. Antimicrobial PEG-amines include PEG-8-10-1-amine, PEG-4-2-1-amine, linear PEG 2-2-1 amine, PEG-8-2-1-amine, PEG-8-40-2-amine and PEG-8-10-2-amine; all of these amines demonstrated dose-dependent inhibition of E. coli growth. PEG amines with the highest amine density, including PEG-8-2-1-amine and PEG-8-10-2-amine, were the most potent inhibitors of E. coli proliferation. These results suggest that the PEG-amines can be utilized as antimicrobial agents.

TABLE 1
Inhibition of E. coli Growth by Various PEG Amines

| | | Volume | Bacterial | Bacteriostatically |
|---|---|---|---|---|
| | | Added | Growth | Effective |
| Example | PEG Amine | (μL) | (cells/mL) | Amount (mg/mL) |
| 1 | P8-10-1/P4- | 0 | 9.50 × 10⁸ | ≧75 |
| | 2-1 (2.7:1 | 20 | 1.13 × 10⁹ | |
| | wt ratio) | 250 | 1.07 × 10⁹ | |
| | 55% solids | 500 | 5.09 × 10⁸ | |
| | | 1000 | 0 | |
| 2 | P8-40-2 | 0 | 9.52 × 10⁸ | ≧75 |
| | 30% solids | 20 | 7.30 × 10⁸ | |
| | | 250 | 4.90 × 10⁸ | |
| | | 500 | 4.55 × 10⁸ | |
| | | 1000 | 2.20 × 10⁸ | |
| 3 | P2-2-1 | 0 | 9.52 × 10⁸ | ≧30 |
| | 50% solids | 20 | 7.90 × 10⁸ | |
| | | 250 | 2.70 × 10⁸ | |
| | | 500 | 1.50 × 10⁵ | |
| | | 1000 | 0 | |
| 4 | P8-10-2 | 0 | 8.22 × 10⁸ | ≧15 |
| | 50% solids | 20 | 7.08 × 10⁸ | |
| | | 250 | 0 | |
| | | 500 | 0 | |
| | | 1000 | 0 | |
| 5 | P8-2-1 | 0 | 8.58 × 10⁸ | ≧15 |
| | 50% solids | 20 | 9.93 × 10⁸ | |
| | | 250 | 0 | |
| | | 500 | 0 | |
| | | 1000 | 0 | |

What is claimed is:

1. A method of inhibiting proliferation of Escherichia coli suspected of being present at an anatomical site on tissue of a human or animal body comprising:
applying to said anatomical site a bacteriostatically effective amount of at least one aminated polyether.

2. The method according to claim 1 wherein the at least one aminated polyether has a number-average molecular weight of about 450 to about 200,000 Daltons.

3. The method according to claim 1 wherein the at least one aminated polyether has a number-average molecular weight of about 2,000 to about 40,000 Daltons.

4. The method according to claim 1 wherein the at least one aminated polyether is selected from the group consisting of a linear polyethylene glycol diamine having a number-average molecular weight of about 2,000 Daltons, an eight arm polyethylene glycol octaamine having a number-average molecular weight of about 10,000 Daltons, an eight arm polyethylene glycol octaamine having a number-average molecular weight of about 2,000 Daltons, a four arm polyethylene glycol tetraamine having a number-average molecular weight of about 2,000 Daltons, an 8-arm polyethylene glycol hexadecaamine having a number-average molecular weight of about 40,000 Daltons, an 8-arm polyethylene glycol hexadecaamine having a number-average molecular weight of about 10,000 Daltons, and any combination thereof.

5. The method according to claim 4 wherein the at least one aminated polyether is an eight arm polyethylene glycol octaamine having a number-average molecular weight of about 2,000 Daltons.

6. The method according to claim 1 wherein the at least one aminated polyether is contained in an aqueous solution or dispersion.

7. The method according to claim 6 wherein the aqueous solution or dispersion is applied to the anatomical site by brushing, pouring, dripping, spraying, wiping, or extrusion using a pipette or a syringe.

8. The method according to claim 6 wherein the aqueous solution or dispersion further comprises at least one additive selected from the group consisting of thickeners, colorants, surfactants, and anti-inflammatory agents.

9. The method according to claim 6 wherein the aqueous solution or dispersion is sterilized.
